(19) United States
(12) Patent Application Publication
(10) Pub. No.: US 2023/0143576 A1
(43) Pub. Date: May 11, 2023

(54) TRIPLY PERIODIC MINIMAL SURFACES FOR 3D PRINTED ORGANS AND TISSUES

(71) Applicant: Lung Biotechnology PBC, Silver Spring, MD (US)

(72) Inventor: Greg Hurst, Bedford, NH (US)

(73) Assignee: Lung Biotechnology PBC, Silver Spring, MD (US)

(21) Appl. No.: 17/981,713

(22) Filed: Nov. 7, 2022

Related U.S. Application Data
(60) Provisional application No. 63/276,914, filed on Nov. 8, 2021.

(51) Int. Cl. A61F 2/00; B33Y 70/00; A61F 2/06; B29C 64/245; C12N 5/00; C12N 5/071

(52) U.S. Cl. CPC A61F 2/0077; B33Y 70/00; A61F 2/062; B29C 64/245; C12N 5/0062; C12N 5/0676; C12N 5/0686

(57) ABSTRACT

An apparatus can include a triply periodic minimal surface. The apparatus can include a 3D scaffold formed from the triply periodic minimal surface. The apparatus can include one or more channels formed by the 3D scaffold. A method of forming a gas exchange unit can include printing a 3D scaffold formed from a triply periodic minimal surface. The 3D scaffold can include a vascular network configured to conduct a fluid. The 3D scaffold can include one or more channels configured to hold a gas. The vascular network can be embedded inside walls of the 3D scaffold. The one or more channels can be positioned between the walls of the 3D scaffold.

### CROSS-REFERENCE TO RELATED APPLICATIONS

This application claims priority to U.S. Provisional Application No. 63/276,914, filed Nov. 8, 2021, the entire contents of which are incorporated herein by reference.

### TECHNICAL FIELD

The present application relates to 3D printed structures.

### BACKGROUND

3D printed structures can mimic the structure and/or function of biological structures.

### SUMMARY

The systems and methods of the present disclosure include a 3D printed structure. The 3D printed structure can achieve gas exchange (e.g., O₂) at physiologically relevant rates.

At least one aspect of the present disclosure is directed to an apparatus. The apparatus can include a 3D scaffold formed from the triply periodic minimal surface. The apparatus can include one or more channels formed by the 3D scaffold.

In some embodiments, the apparatus includes a vascular network embedded within the 3D scaffold. In some embodiments, the vascular network has a thickness in a range of 5 μm to 500 μm. In some embodiments, the vascular network and the triply periodic minimal surface are separated by an interface distance in a range of 1 μm to 400 μm. In some embodiments, the apparatus includes a gas exchange chip. The 3D scaffold can include a gas exchange scaffold seeded with biological materials. The gas exchange scaffold seeded with the biological materials can be disposed within the gas exchange chip.

In some embodiments, the one or more channels include airways. The triply periodic minimal surface can include a gyroid. The triply periodic minimal surface can include a Batwing. The triply periodic minimal surface can include a Fischer S. The 3D scaffold can have a thickness in a range of 15 μm to 700 μm. In some embodiments, the 3D scaffold includes a 3D printed scaffold and the 3D printed scaffold replicates the structure or function, at least in part, of at least one of a pancreas, a kidney, a lung, a liver, and bone. The 3D scaffold can include a cell bioreactor that is permeable to at least one of gas, fluid, small molecules, cytokines, or growth factors. In some embodiments, the triply periodic minimal surface can be at least one of an internal mechanical support structure or an external mechanical support structure for a 3D printed scaffold.

Another aspect of the present disclosure is directed to a method of forming a gas exchange unit. The method can include printing a 3D scaffold formed from a triply periodic minimal surface. The 3D scaffold can include a vascular network configured to conduct a fluid. The 3D scaffold can include one or more channels configured to hold a gas. The vascular network can be embedded inside walls of the 3D scaffold. The one or more channels can be positioned between the walls of the 3D scaffold.

In some embodiments, the method can include disposing the 3D scaffold into a gas exchange chip. The triply periodic minimal surface can include a gyroid. The triply periodic minimal surface can include a Batwing. The triply periodic minimal surface can include a Fischer S. The vascular network can have a thickness in a range of 5 μm to 500 μm. The vascular network and the triply periodic minimal surface can be separated by an interface distance in a range of 1 μm to 400 μm.

Those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

### BRIEF DESCRIPTION OF THE DRAWINGS

The details of one or more implementations of the subject matter described in this specification are set forth in the accompanying drawings and the description below. Other features, aspects, and advantages of the subject matter will become apparent from the description, the drawings, and the claims.

FIG. 1A illustrates a schematic of a gyroid, according to an embodiment.

FIG. 1B illustrates a schematic of a Batwing, according to an embodiment.

FIG. 1C illustrates a schematic of a Fischer S, according to an embodiment.

FIG. 2 illustrates a schematic of a 3×3×3 array of gyroid unit cells, according to an embodiment.

FIG. 3 illustrates a schematic of a walled Fischer S unit, according to an embodiment.

FIG. 4 illustrates a table of properties of minimal surfaces, according to an embodiment.

FIG. 5A illustrates a schematic of vascular nets embedded in a scaffold formed from a gyroid, according to an embodiment.

FIG. 5B illustrates a schematic of vascular nets embedded in a scaffold formed from a gyroid with transparent walls, according to an embodiment.

FIG. 5C illustrates a schematic of vascular nets embedded in a scaffold formed from a Batwing, according to an embodiment.

FIG. 5D illustrates a schematic of vascular nets embedded in a scaffold formed from a Batwing with transparent walls, according to an embodiment.

FIG. 5E illustrates a schematic of vascular nets embedded in a scaffold formed from a Fischer S, according to an embodiment.

FIG. 5F illustrates a schematic of vascular nets embedded in a scaffold formed from a Fischer S with transparent walls, according to an embodiment.

FIG. 6 illustrates a schematic of a 3D chip with Fischer S walls and vasculature, according to an embodiment.

FIG. 7A illustrates a top view of the Fischer S walls shown in FIG. 6, according to an embodiment.

FIG. 7B illustrates a top view of the vasculature shown in FIG. 6, according to an embodiment.

FIG. 8 illustrates a cross-sectional view of the 3D chip shown in FIG. 6 taken along plane A-A, according to an embodiment.

FIG. 9 illustrates a cross-sectional view of the 3D chip shown in FIG. 6 taken along plane B-B, according to an embodiment.

FIG. 10 illustrates a schematic of a 3D chip model showing distances to one or more airway, according to an embodiment.

FIG. 11 illustrates a schematic of a model of a giant Fischer 3D chip, according to an embodiment.

FIG. 12 illustrates a schematic of a giant Fischer 3D chip, according to an embodiment.

FIG. 13 illustrates a schematic of a model of a Fischer block 3D chip, according to an embodiment.

FIG. 14 illustrates a schematic of a Fischer block 3D chip, according to an embodiment.

FIG. 15 illustrates a table of gas exchange data for the giant Fischer 3D chip and the Fischer block 3D chip, according to an embodiment.

Like reference numbers and designations in the various drawings indicate like elements.

### DETAILED DESCRIPTION

The systems and methods of the present disclosure can be used to generate 3D printed scaffolds corresponding to biological structure or function. For example, the 3D printed scaffolds can correspond to the structure or function of the pancreas, kidney, lung, liver, bone, or any organ which is made up of similar repeating structural units. The 3D printed scaffold does not necessarily need to be identical in structure or function to the corresponding naturally-occurring biological structure or function. In some embodiments, the 3D printed scaffold can be based on a model of the corresponding biological structure or function, and the 3D printed scaffold can comprise repeating structural of functional units of the corresponding organ. Some embodiments include 3D printed scaffolds corresponding to human lungs and kidneys. The systems and methods of the present disclosure can be used to print a scaffold which serves as a cell bioreactor that is permeable to gas and fluid. Additionally, the systems and methods of the present disclosure can be used as support structures during the print process of 3D printed objects.

A minimal surface can include a surface that is locally area-minimizing (e.g., a small piece has the smallest possible area for a surface spanning the boundary of that piece). Soap films are examples of minimal surfaces. Minimal surfaces can have zero mean curvature (e.g., the sum of the principal curvatures at each point is zero). Minimal surfaces can have a crystalline structure (e.g., repeating themselves in three dimensions) and/or be triply periodic.

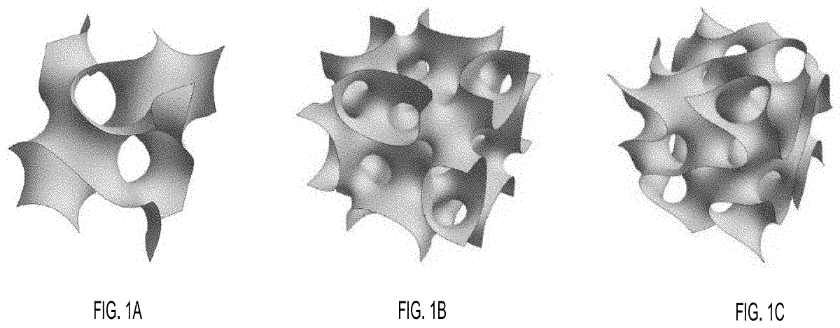

FIGS. 1A-1C illustrate some exemplary triply periodic minimal surfaces (TPMS). FIG. 1A illustrates a schematic of a gyroid (e.g., gyroid surface). An example of a TPMS can include the gyroid. FIG. 1B illustrates a schematic of a Batwing (e.g., Batwing surface). Another example of a TPMS can include the Batwing. FIG. 1C illustrates a schematic of a Fischer S (e.g., Fischer S surface). A TPMS can also include the Fischer S. Other examples of triply periodic minimal surfaces include Schwarz' P surface, Batwing surfaces, Schwarz' D surface, Schoen's gyroid surface, the complementary D surface, Neovius' surface, the N14 surface, the N26 surface, the N38 surface, complementary P surfaces, starfish surfaces, disphenoid surfaces, the p3a surface, hybrid surfaces, Schwarz' H surface, Schoen's RII surface, Schoen's RIII surface, Schoen's I-6 surface, Schoen's I-8 surface, Schoen's I-9 surface, Schwarz' CLP surface, Schoen's F-RD surface, Schoen's hybrid-1[P,F-RD] surface, Schoen's GW surface, Schoen's I-WP surface, Schoen's S′-S″ surface, Schoen's S′-S″|P surface, Schoen's H′-T surface, Schoen's H″-R surface, Schoen's T′-R′ surface, Schoen's H′-T|H″-R surface, Schoen's T′-R′|H′-T surface, Schoen's H″-R|T′-R′ surface, Schoen's O,C-TO surface, Schoen's F-RD(r) surface, Schoen's I-WP(r) surface, Schoen's Manta surface of Genus 19, Schoen's Manta surface of Genus 35, Schoen's Manta surface of Genus 51, triplane surfaces, hexplane surfaces, Fisher-Koch S surface, Fisher-Koch C(S) surface, Fisher-Koch Y surface, and Fisher-Koch C(Y) surface. Any TPMS, including these specific examples of TPMS, can be used alone or in combination for 3D printed scaffolds according to the present disclosure.

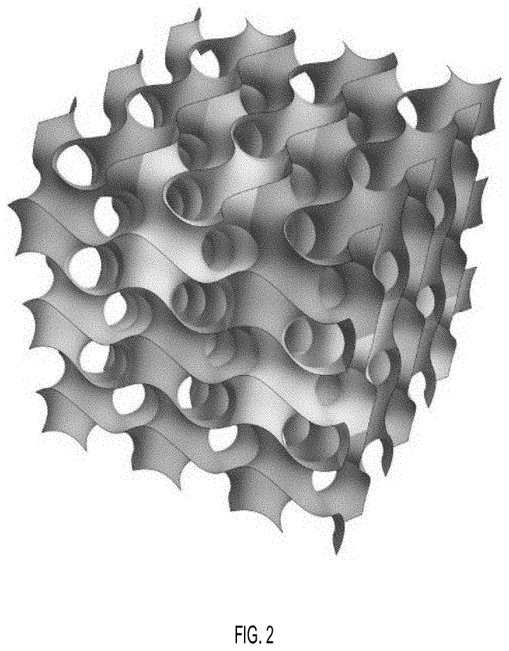

FIG. 2 illustrates a schematic of a 3×3×3 array of gyroid unit cells. A TPMS can be periodic in all three cardinal directions. TPMS can be seamlessly tiled, as shown in FIG. 2. The 3×3×3 array of gyroid unit cells can include 27 gyroid unit cells.

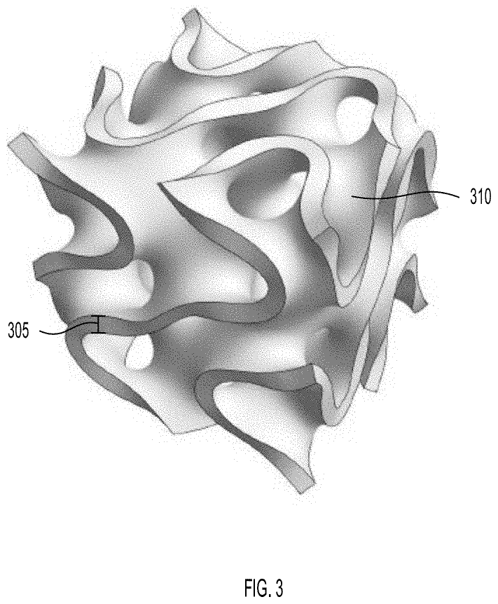

FIG. 3 illustrates a schematic of a walled Fischer S unit. The walled Fischer S unit can include a thickness 305 (e.g., prescribed thickness). The walled Fischer S unit can include a surface 310 (e.g., wall, tissue, etc.). TPMS are surfaces and therefore have zero volume. To create a volume (e.g., 3D unit, 3D scaffold, 3D printed structure etc.), one can extrude along the surface normals to turn the surfaces (e.g., sheets) into walls with a prescribed thickness. The 3D printed structures can mimic the structure and/or function of biological structures. The 3D printed structures can be used to model physiological processes. The walls (e.g., walls with thickness) can model tissue. The channels in between the walls can model airways. With vasculature inside the walls, the 3D scaffolds can be considered gas exchange units (e.g., mock-alveoli). The tissue thickness and airway channel size can be customizable. The tissue thickness can be selected. The unit cell can be scaled up until the airway channels are of the desired (e.g., target) size. Per Fick's law of diffusion, thinner walls can allow for better gas exchange.

Every point on a TPMS can include a saddle point (e.g., there is a cross section where the point is a maxima and a perpendicular cross section is a minima). Since every point on a TPMS can include a saddle point, there are no local minima for any given orientation of the model. During a 3D printing process, no matter the orientation of the model, there will not be any unprintable cross sections of the model.

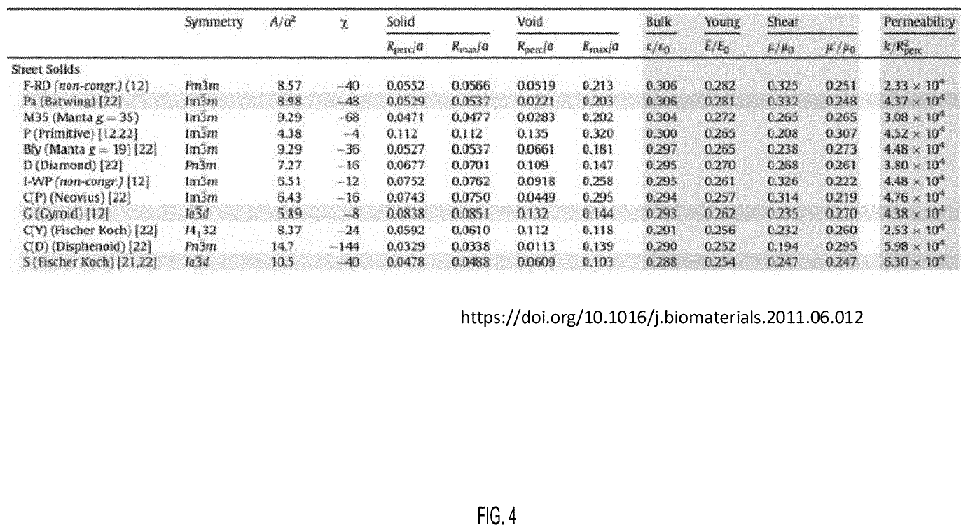

FIG. 4 illustrates a table of properties of minimal surfaces. Per Fick's law of diffusion, having a large interface area between the airway and the vasculature can allow for better gas exchange. The labrynthian airway channels found in TPMS can provide a large surface area per unit volume. For a 1 mm³ unit cell of thin-walled gyroid, the surface area can be 5.89 mm² (square mm). For a 1 mm³ (cubic mm) unit cell of thin-walled Batwing, the surface area can be 8.98 mm². For a 1 mm³ unit cell of thin-walled Fischer S, the surface area can be 10.5 mm². The TPMS (e.g., a 3D scaffold based on a TPMS) can be printed in a hydrogel. For example, the TPMS can be 3D printed in the hydrogel. The TPMS can withstand forces (e.g., compressive forces, elastic forces, shear forces, etc.) experienced during the printing process (e.g., 3D printing process) and/or ventilation process. A collection of TPMS can exhibit mechanical stiffness quasi-isotropically. The moduli values for the gyroid, Batwing, and Fischer S can be near the Hashin-Shtrikman (HS) bound (e.g., within 5%, 10%, 15%, or 20% of the HS bound). The moduli values for the gyroid, Batwing, and Fischer S can be ⅓ of the HS bound.

The Batwing can have a bulk modulus in a range of 0.204 K/K₀ to 0.408 K/K₀ (e.g., 0.306 K/K₀). The Batwing can have a Young's modulus in a range of 0.187 Ē/E₀ to 0.375 Ē/E₀ (e.g., 0.281 Ē/E₀). The Batwing can have a shear modulus in a range of 0.221 μ/μ₀ to 0.443 μ/μ₀ (e.g., 0.332 μ/μ₀). The Batwing can have a shear modulus in a range of 0.165 μ′/μ₀ to 0.331 μ′/μ₀ (e.g., 0.248 μ′/μ₀).

The gyroid can have a bulk modulus in a range of 0.195 K/K₀ to 0.391 K/K₀ (e.g., 0.293 K/K₀). The gyroid can have a Young's modulus in a range of 0.175 Ē/E₀ to 0.349 Ē/E₀ (e.g., 0.262 Ē/E₀). The gyroid can have a shear modulus in a range of 0.156 μ/μ₀ to 0.313 μ/μ₀ (e.g., 0.235 μ/μ₀). The gyroid can have a shear modulus in a range of 0.180 μ′/μ₀ to 0.360 μ′/μ₀ (e.g., 0.270 μ′/μ₀).

The Fischer S can have a bulk modulus in a range of 0.192 K/K₀ to 0.384 K/K₀ (e.g., 0.288 K/K₀). The Fischer S can have a Young's modulus in a range of 0.169 Ē/E₀ to 0.339 Ē/E₀ (e.g., 0.254 Ē/E₀). The Fischer S can have a shear modulus in a range of 0.165 μ/μ₀ to 0.329 μ/μ₀ (e.g., 0.247 μ/μ₀). The Fischer S can have a shear modulus in a range of 0.165 μ′/μ₀ to 0.329 μ′/μ₀ (e.g., 0.247 μ′/μ₀).

Permeability can be part of the proportionality constant in Darcy's law, which relates flow rate and fluid physical properties (e.g. viscosity), to a pressure gradient applied to porous media. High permeability can be desired for better gas exchange, as permeability relates to spread of flow throughout media. FIG. 4 shows permeability for various TPMS at a given length scale. The Batwing unit cell has a permeability of 4.37×10⁴ k/R_{perc}². The gyroid unit cell has a permeability of 4.38×10⁴ k/R_{perc}². The Fischer S unit cell has a permeability of 6.30×10⁴ k/R_{perc}².

Gas exchange can occur between two media across a barrier interface. In the case of walled TPMS (e.g., 3D scaffold), the tissue walls can have air channels on both sides and so embedding vasculature inside the walls can give such a barrier interface between blood and air. The blood can have two separate air channels in which gas exchange can occur. High surface area TPMS can allow for dense vascular networks to be formed. This net can become denser as the diameter of the vasculature decreases. This can be important for gas exchange, as Fick's law shows that more area for diffusion increases gas exchange.

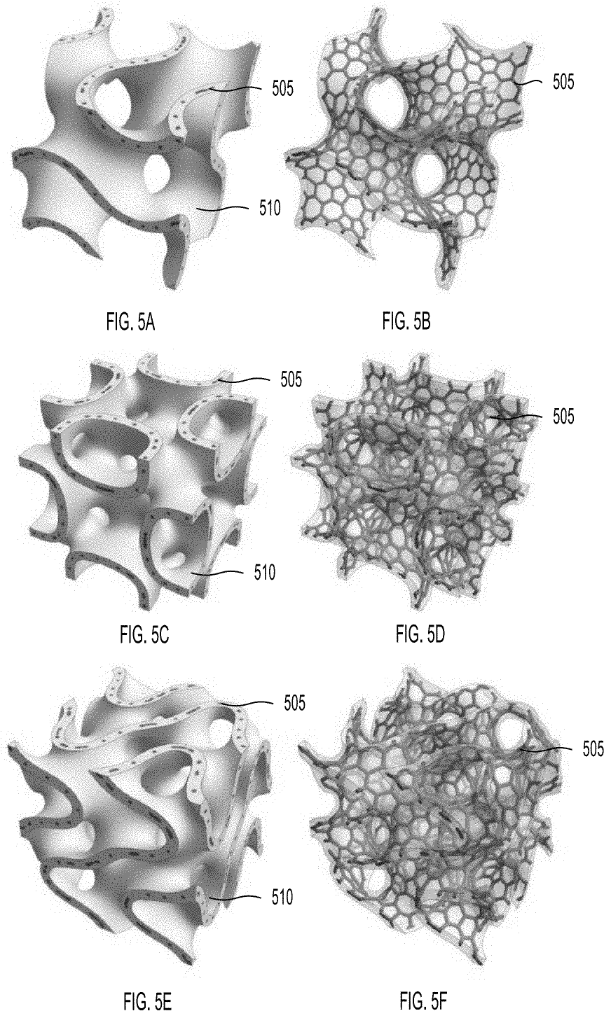

FIGS. 5A-5F illustrate examples of dense vascular nets (e.g., vasculature 505) embedded in a scaffold 510 formed from a gyroid, a Batwing, and a Fischer S. FIG. 5A illustrates a schematic of vascular nets (e.g., vasculature 505, vascular network, etc.) embedded in the scaffold 510 formed from a gyroid (e.g., gyroid scaffold, walled gyroid, etc.). The vasculature 505 can be embedded within the walls of the scaffold 510. FIG. 5B illustrates a schematic of vascular nets (e.g., vasculature 505) embedded in the scaffold 510 formed from a gyroid with transparent walls. The transparent walls can reveal a dense network of vasculature.

FIG. 5C illustrates a schematic of vascular nets (e.g., vasculature 505) embedded in the scaffold 510 formed from a Batwing surface (e.g., Batwing scaffold, walled Batwing, etc.). The vasculature 505 can be embedded within the walls of the scaffold 510. FIG. 5D illustrates a schematic of vascular nets (e.g., vasculature 505) embedded in the scaffold 510 formed from a Batwing with transparent walls. The transparent walls can reveal a dense network of vasculature.

FIG. 5E illustrates a schematic of vascular nets (e.g., vasculature 505) embedded in the scaffold 510 formed from a Fischer S surface (e.g., Fischer S scaffold, walled Fischer S, etc.). The vasculature 505 can be embedded within the walls of the scaffold 510. FIG. 5F illustrates a schematic of vascular nets (e.g., vasculature 505) embedded in the scaffold 510 formed from a Fischer S with transparent walls. The transparent walls can reveal a dense network of vasculature.

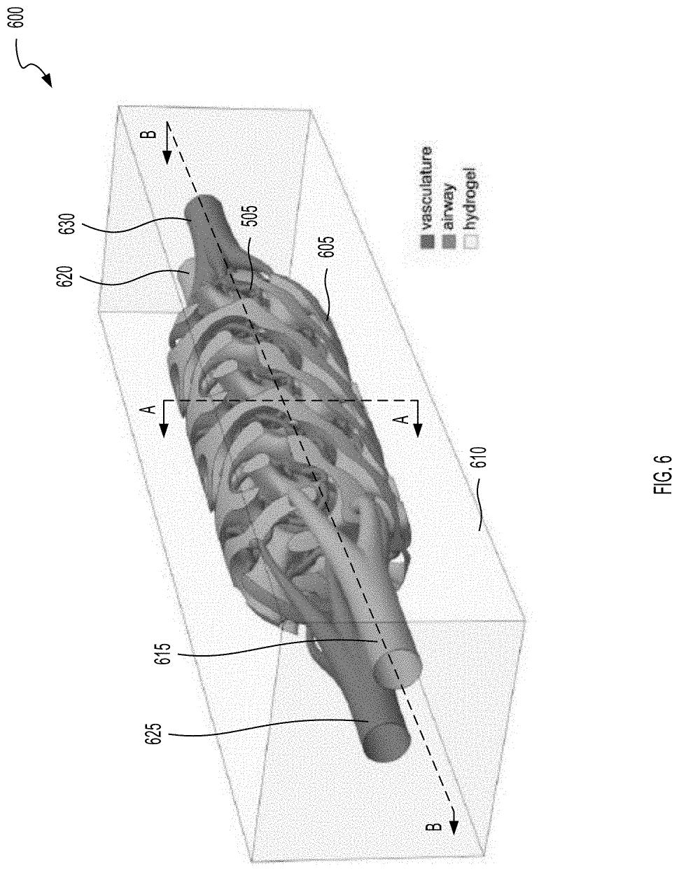

FIG. 6 illustrates a schematic of a 3D chip 600 with Fischer S walls and vasculature. The 3D chip 600 can include walls based on TPMS. The 3D chip 600 (e.g., 3D gas exchange chip, apparatus) can include one or more airways 605 (e.g., airways, air channels, etc.) enclosed by the walls (e.g., tissue). The 3D chip 600 can include vasculature 505 embedded inside the walls. The Fischer S TPMS can be tiled to fit into a capsule shape. The one or more airways 605 can be fed by a bifurcating inlet. The 3D chip 600 can include an airway inlet 615. The 3D chip 600 can include one or more airway inlets. The 3D chip 600 can include an airway outlet 620. The 3D chip 600 can include one or more airway outlets. The TPMS can be inflated and/or deflated. The 3D chip 600 can include a vasculature inlet 625. The 3D chip 600 can include one or more vasculature inlets. The 3D chip 600 can include a vasculature outlet 630. The 3D chip 600 can include one or more vasculature outlets. The vasculature 505 can be fed from an inlet (e.g., bifurcating inlet, trifurcating inlet, 4-furcating inlet, 5-furcating inlet, 6-furcating inlet, etc.). The vasculature can exit through a 6-furcating outlet (e.g., bifurcating outlet, trifurcating outlet, 4-furcating outlet, 5-furcating outlet, 6-furcating outlet, etc.). The 3D chip 600 can include a hydrogel 610 (e.g., hydrogel matrix, matrix, etc.). The 3D chip 600 can be a 3D structure, as opposed to a 2D or planar structure. The 3D structure can provide more surface area per unit volume, which can lead to improved gas exchange over 2D or planar structures. The 3D chip 600 can have a width and a height. The width of the 3D chip 600 and the height of the 3D chip 600 can be the same order of magnitude. In contrast, a 2D or planar structure can have a width that is substantially greater than the height. For example, the 2D or planar structure can have a width that is one or more orders of magnitude greater than the height.

The TPMS can include at least one of an internal mechanical support structure or an external mechanical support structure for a 3D printed scaffold. For example, a support structure based on the TPMS can serve as mechanical support for a 3D printed scaffold. The support structure can prevent part deformation, secure a part to a printing bed, and/or ensure that parts are attached to the main body of the printed part.

The 3D chip 600 can include a triply periodic minimal surface. The 3D chip can include the scaffold 510 (e.g., 3D scaffold, 3D printed scaffold, etc.) formed from the triply periodic minimal surface. The triply periodic minimal surface can include a gyroid. The triply periodic minimal surface can include a Batwing. The triply periodic minimal surface can include a Fischer S. The 3D scaffold can include the one or more airways 605. The 3D chip can include one or more channels formed by the 3D scaffold. The one or more channels can include the one or more airways 605. The vascular network can be embedded within the 3D scaffold. For example, the vasculature 505 can be enclosed by the walls of the scaffold 510. The 3D printed scaffold can model at least one of an organ, an organoid, or a fragment of an organ. For example, the 3D printed scaffold can model a kidney, a heart, a liver, a lung, a spleen, a brain, a vessel, a gallbladder, a stomach, a pancreas, a bladder, bone, skeletal bone, cartilage, skin, a hair follicle, an intestine, a muscle, a larynx, a pharynx, or any organ which is made of similar repeating structural units. The 3D scaffold can include a cell bioreactor that is permeable to at least one of gas, fluid, small molecules, cytokines, or growth factors.

The apparatus can include a gas exchange chip (e.g., 3D chip 600). The gas exchange chip can exchange gases (e.g., O₂). The 3D scaffold can include a gas exchange scaffold (e.g., 3D scaffold) seeded with biological materials (e.g., islet cells, epithelial cells, osteoblasts, osteoclasts, osteocytes, etc.). The gas exchange scaffold seeded with the biological materials can be disposed within the gas exchange chip.

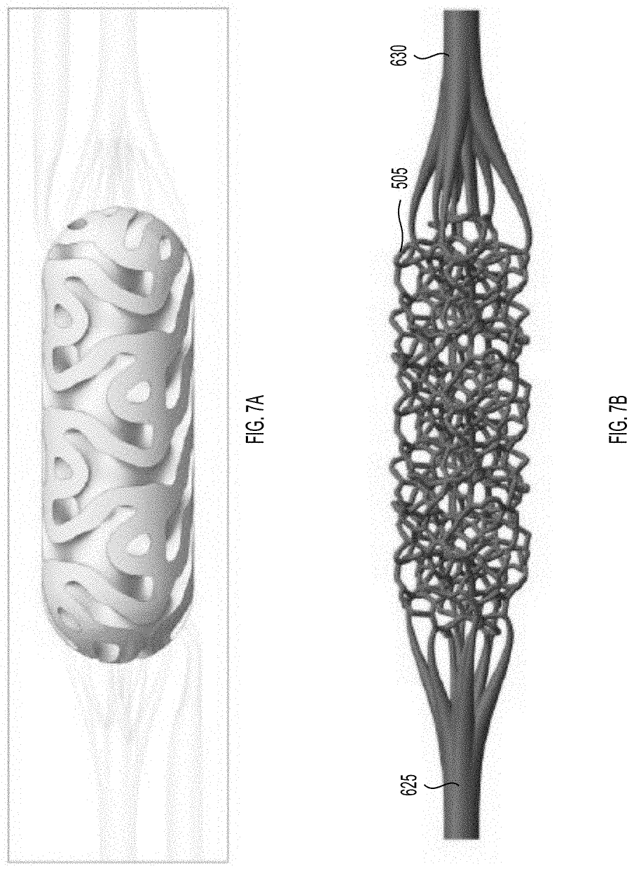

FIG. 7A illustrates a top view of the Fischer S walls (e.g., tissue) shown in FIG. 6. The air channels can be between the walls. For example, the air channels can be formed by the walls. The vasculature 505 can be embedded inside the walls. FIG. 7B illustrates a top view of the vasculature 505 shown in FIG. 6. The vasculature 505 can be located in the walls (e.g., Fischer S walls). The vasculature 505 can include the vasculature inlet 625. The vasculature 505 can include the vasculature outlet 630.

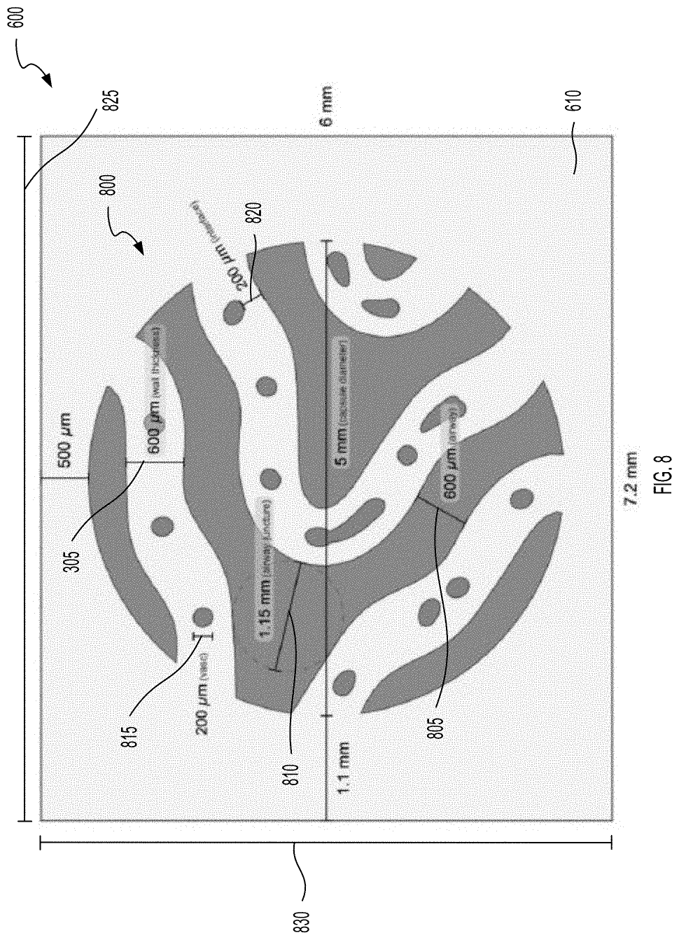

FIG. 8 illustrates a cross-sectional view of the 3D chip 600 shown in FIG. 6 taken along plane A-A. The 3D chip 600 can have a width 825 of 7.2 mm and a height 830 of 6 mm. The 3D chip 600 can include a capsule 800 (e.g., 3D scaffold, gas exchange scaffold, etc.). The 3D scaffold can have a thickness in a range of 15 μm to 700 μm (e.g., 15 μm, 50 μm, 75 μm, 100 μm, 125 μm, 150 μm, 175 μm, 200 μm, 225 μm, 250 μm, 275 μm, 300 μm, 325 μm, 350 μm, 375 μm, 400 μm, 500 μm, 525 μm, 550 μm, 575 μm, 600 μm, 625 μm, 650 μm, 675 μm, 700 μm, inclusive). The capsule 800 can have a diameter (e.g., capsule diameter) of 5 mm. A distance from the capsule 800 and an edge of the 3D chip 600 can be in a range from 500 μm to 1.1 mm. The capsule 800 can be embedded in the hydrogel 610. The capsule 800 can include the thickness 305 (e.g., wall thickness). The wall thickness can include the thickness of TPMS walls. The wall thickness can be, for example, 600 μm. The capsule 800 can include an airway thickness 805. The airway thickness 805 can include the thickness (e.g., size) of one or more airways channels. The airway thickness 805 can be, for example, 600 μm. The capsule 800 can include an airway juncture thickness 810. The airway juncture thickness 810 can include the thickness (e.g., size) of one or more airway channels where the airways fork and/or bifurcate. The airway juncture thickness 810 can be, for example, 1.15 mm.

The capsule 800 can include a vasculature diameter 815. The vasculature diameter 815 (e.g., thickness of the vascular network) can include the diameter of vasculature in the TPMS walls. The vasculature diameter 815 can be, for example, 200 μm. The vascular network can have a thickness in a range of 5 μm to 500 μm (e.g., 5 μm, 10 μm, 20 μm, 50 μm, 100 μm, 200 μm, 300 μm, 400 μm, 500 μm, inclusive). The capsule 800 can include an interface distance 820. The interface distance 820 can include the distance from the vasculature to the one or more airways 605. The interface distance 820 can be, for example, 200 μm. The vascular network and the triply periodic minimal surface can be separated by the interface distance 820. The interface distance 820 can be in a range of 1 μm to 400 μm (e.g., 1 μm, 5 μm, 10 μm, 25 μm, 50 μm, 100 μm, 200 μm, 300 μm, 400 μm, inclusive). The capsule 800 can have a width and a height. The width of the capsule 800 and the height of the capsule 800 can be the same order of magnitude.

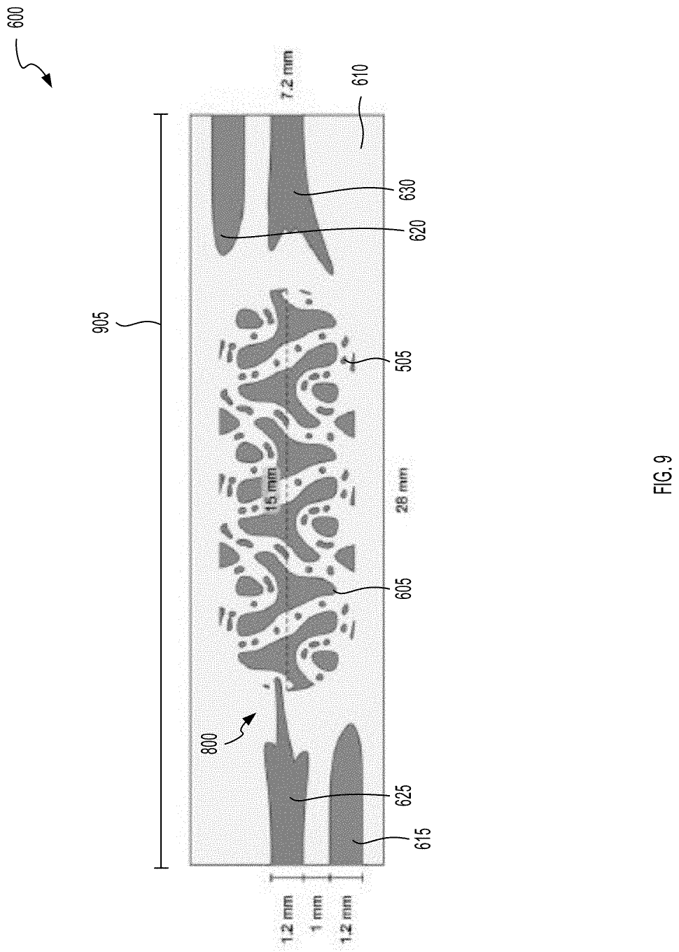

FIG. 9 illustrates a cross-sectional view of the 3D chip 600 shown in FIG. 6 taken along plane B-B. The 3D chip 600 can have a width of 7.2 mm and a length 905 of 28 mm. The chip 600 can include the capsule 800. The capsule 800 can be embedded in the hydrogel 610. The capsule 800 can have a length (e.g., capsule length) of 15 mm. For an interface distance L and vasculature 505 with diameter D, the distance from a point on the vasculature 505 to the one or more airways 605 can be between L and L+D/2. The capsule 800 can include the airway inlet 615. The capsule 800 can include the airway outlet 620. The capsule 800 can include the vasculature inlet 625. The capsule 800 can include the vasculature outlet 630.

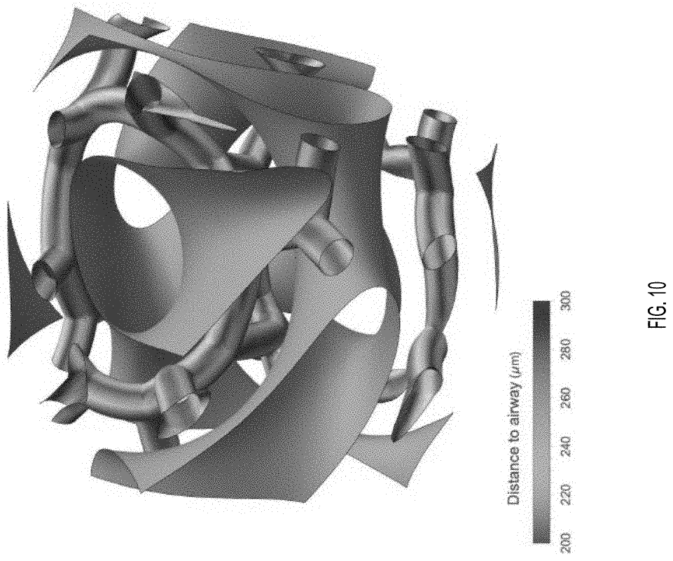

FIG. 10 illustrates a schematic of a 3D chip model showing distance to the one or more airways 605. The distance from a point on the vasculature 505 to the one or more airways 605 can be in a range of 200 μm and 300 μm (e.g., 200 μm, 205 μm, 210 μm, 215 μm, 220 μm, 225 μm, 230 μm, 235 μm, 240 μm, 245 μm, 250 μm, 255 μm, 260 μm, 265 μm, 270 μm, 275 μm, 280 μm, 285 μm, 290 μm, 295 μm, 300 μm, inclusive).

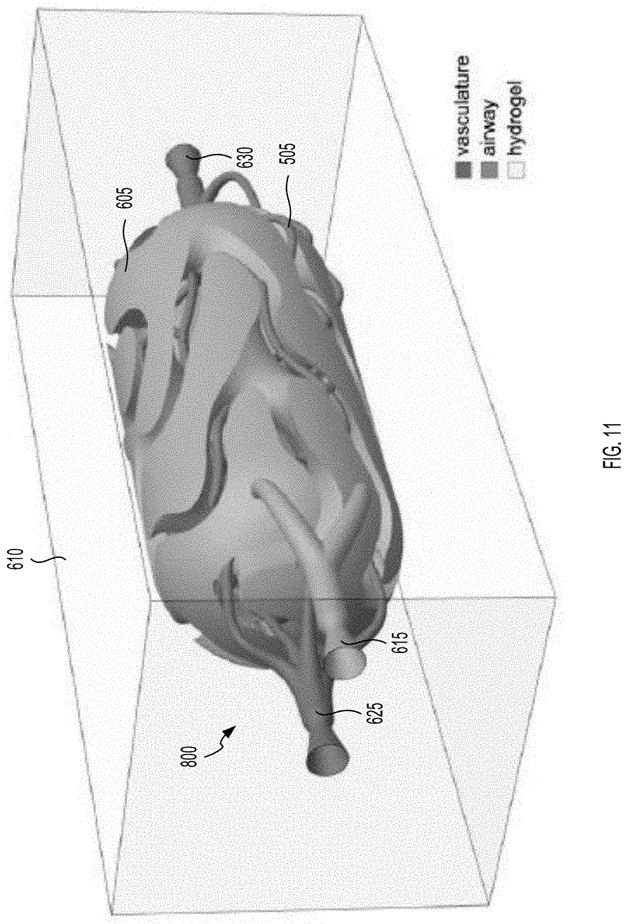

FIG. 11 illustrates a schematic of a model (e.g., computer render, computer rendered image, etc.) of a giant Fischer 3D chip (e.g., giant Fischer). The model can include the vasculature 505. The model can include the one or more airways 605. The capsule 800 can be embedded in the hydrogel 610. The vasculature 505 and the one or more airways 605 can be embedded in the hydrogel 610. The capsule 800 can include the airway inlet 615. The capsule 800 can include the airway outlet 620 (not shown). The capsule 800 can include the vasculature inlet 625. The capsule 800 can include the vasculature outlet 630.

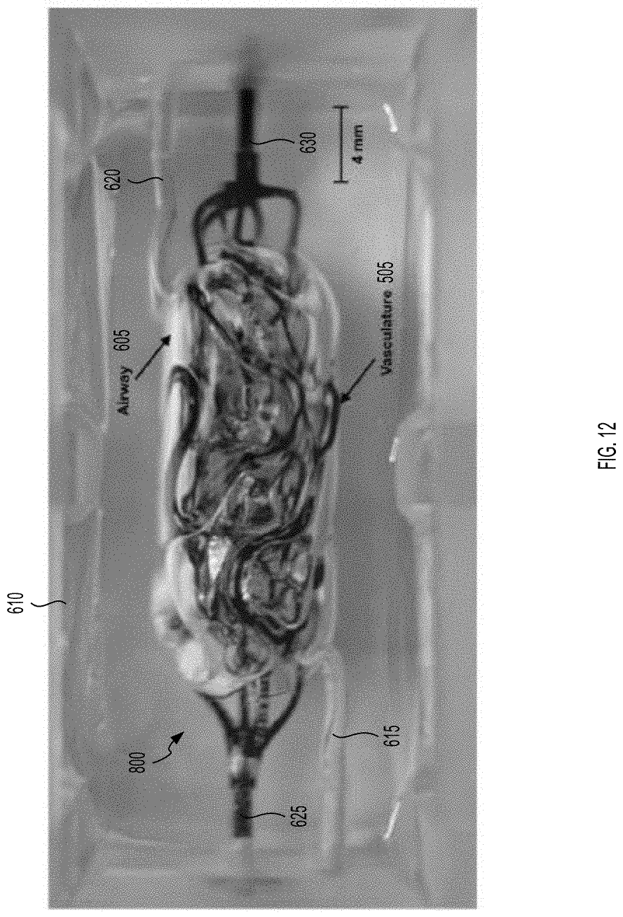

FIG. 12 illustrates a schematic of a giant Fischer 3D chip. The giant Fischer 3D chip is shown after gas exchange (e.g., ventilated and perfused with blood). The capsule 800 can be embedded in the hydrogel 610. The vasculature 505 and the one or more airways 605 can be embedded in the hydrogel 610. The capsule 800 can include the airway inlet 615. The capsule 800 can include the airway outlet 620. The capsule 800 can include the vasculature inlet 625. The capsule 800 can include the vasculature outlet 630.

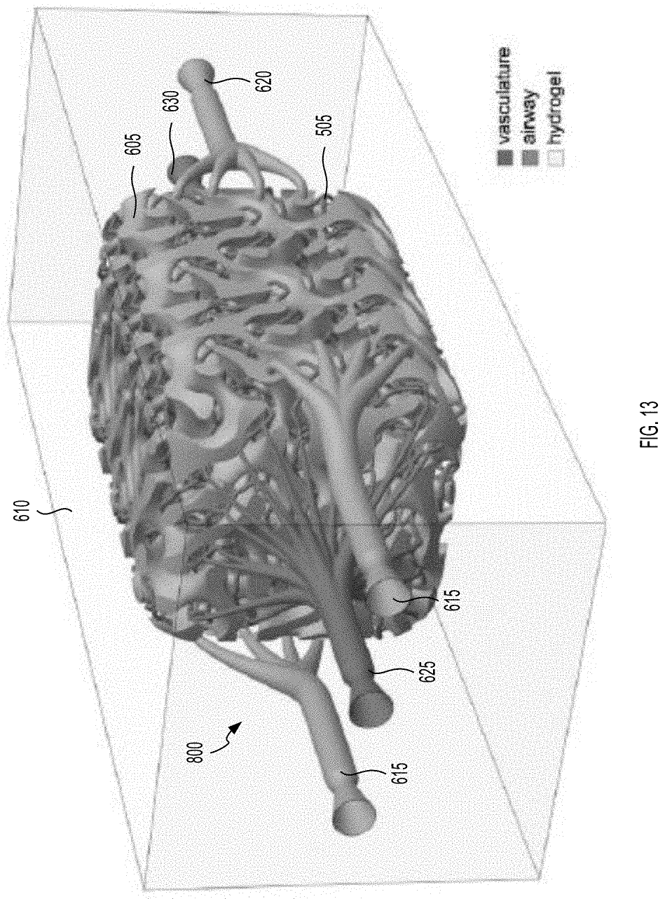

FIG. 13 illustrates a schematic of a model (e.g., computer render, computer rendered image, etc.) of a Fischer block 3D chip (e.g., Fischer block). The model can include the vasculature 505. The model can include the one or more airways 605. The capsule 800 can be embedded in the hydrogel 610. The vasculature 505 and the one or more airways 605 can be embedded in the hydrogel 610. The capsule 800 can include the airway inlet 615. For example, the capsule 800 can include two airway inlets. The capsule 800 can include the airway outlet 620. The capsule 800 can include the vasculature inlet 625. The capsule 800 can include the vasculature outlet 630.

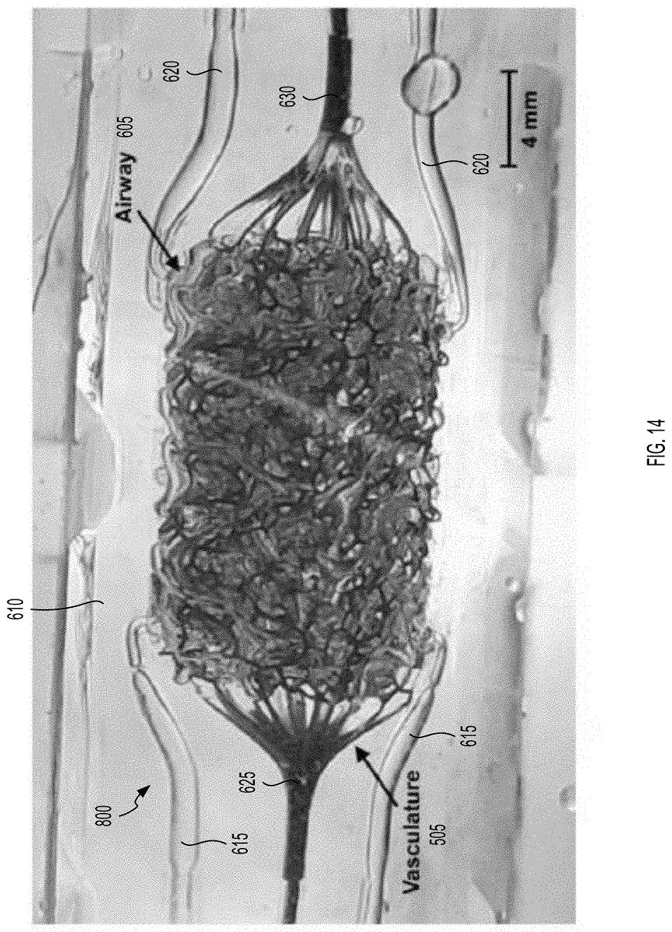

FIG. 14 illustrates a schematic of a Fischer block 3D chip. The Fischer block 3D chip is shown after gas exchange (e.g., ventilated and perfused with blood). The capsule 800 can be embedded in the hydrogel 610. The vasculature 505 and the one or more airways 605 can be embedded in the hydrogel 610. The capsule 800 can include the airway inlet 615. For example, the capsule 800 can include two airway inlets. The capsule 800 can include the airway outlet 620. The capsule 800 can include the vasculature inlet 625. The capsule 800 can include the vasculature outlet 630.

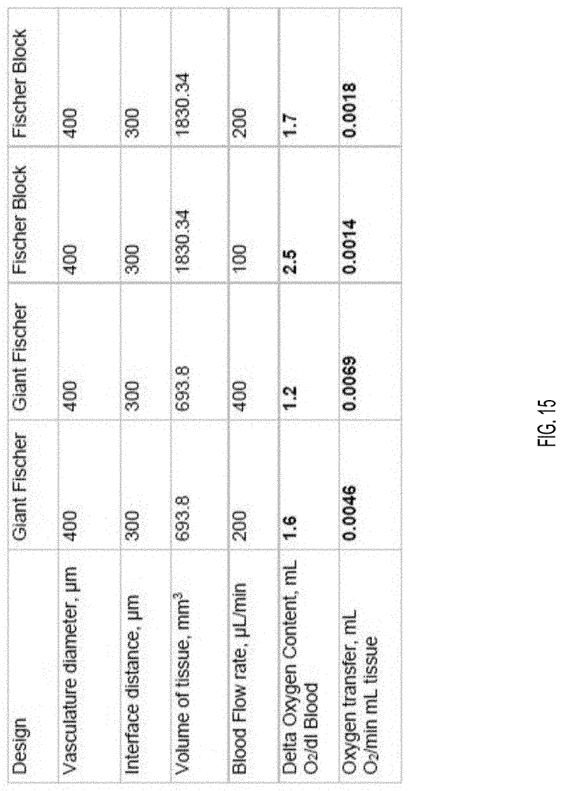

FIG. 15 illustrates a table of gas exchange data for the giant Fischer 3D chip and the Fischer block 3D chip. The giant Fischer 3D chip can have a vascular diameter of 400 μm. The giant Fischer 3D chip can have an interface distance of 300 μm. The giant Fischer 3D chip can have a volume of tissue of 693.8 mm³. The giant Fischer 3D chip can have a blood flow rate of 200 μL/min. The giant Fischer 3D chip can have a delta oxygen content of 1.6 mL O₂/dl blood. The giant Fischer 3D chip can have an oxygen transfer of 0.0046 mL O₂/dl tissue.

The giant Fischer 3D chip can have a vascular diameter of 400 μm. The giant Fischer 3D chip can have an interface distance of 300 μm. The giant Fischer 3D chip can have a volume of tissue of 693.8 mm³. The giant Fischer 3D chip can have a blood flow rate of 400 μL/min. The giant Fischer 3D chip can have a delta oxygen content of 1.2 mL O₂/dl blood. The giant Fischer 3D chip can have an oxygen transfer of 0.0069 mL O₂/dl tissue.

The Fischer block 3D chip can have a vascular diameter of 400 μm. The Fischer block 3D chip can have an interface distance of 300 μm. The Fischer block 3D chip can have a volume of tissue of 1830.34 mm³. The Fischer block 3D chip can have a blood flow rate of 100 μL/min. The Fischer block 3D chip can have a delta oxygen content of 2.5 mL O₂/dl blood. The Fischer block 3D chip can have an oxygen transfer of 0.0014 mL O₂/dl tissue.

The Fischer block 3D chip can have a vascular diameter of 400 μm. The Fischer block 3D chip can have an interface distance of 300 μm. The Fischer block 3D chip can have a volume of tissue of 1830.34 mm³. The Fischer block 3D chip can have a blood flow rate of 200 μL/min. The Fischer block 3D chip can have a delta oxygen content of 1.7 mL O₂/dl blood. The Fischer block 3D chip can have an oxygen transfer of 0.0018 mL O₂/dl tissue.

A method of forming a gas exchange unit can include printing a 3D scaffold formed from a triply periodic minimal surface. The 3D scaffold can be formed from the triply periodic minimal surface. The triply periodic minimal surface can include at least one of a gyroid, a Batwing, or a Fischer S. The triply periodic minimal surface can be at least one of an internal mechanical support structure or an external mechanical support structure for a 3D printed scaffold.

An example of a TPMS can include the Fischer S. Other examples of triply periodic minimal surfaces can include Schwarz' P surface, Batwing surfaces, Schwarz' D surface, Schoen's gyroid surface, the complementary D surface, Neovius' surface, the N14 surface, the N26 surface, the N38 surface, complementary P surfaces, starfish surfaces, disphenoid surfaces, the p3a surface, hybrid surfaces, Schwarz' H surface, Schoen's RII surface, Schoen's RIII surface, Schoen's 1-6 surface, Schoen's 1-8 surface, Schoen's 1-9 surface, Schwarz' CLP surface, Schoen's F-RD surface, Schoen's hybrid-1[P,F-RD] surface, Schoen's GW surface, Schoen's I-WP surface, Schoen's S′-S″ surface, Schoen's S′-S″|P surface, Schoen's H′-T surface, Schoen's H″-R surface, Schoen's T′-R′ surface, Schoen's H′-T|H″-R surface, Schoen's T′-R′|H′-T surface, Schoen's H″-R|T′-R′ surface, Schoen's O,C-TO surface, Schoen's F-RD(r) surface, Schoen's I-WP(r) surface, Schoen's Manta surface of Genus 19, Schoen's Manta surface of Genus 35, Schoen's Manta surface of Genus 51, triplane surfaces, hexplane surfaces, Fisher-Koch S surface, Fisher-Koch C(S) surface, Fisher-Koch Y surface, and Fisher-Koch C(Y) surface.

The 3D scaffold can include a vascular network (e.g., vasculature, vascular nets, etc.). The vascular network can conduct a fluid. The vascular network can have a thickness in a range of 5 μm to 500 μm (e.g., 5 μm, 10 μm, 20 μm, 50 μm, 100 μm, 200 μm, 300 μm, 400 μm, 500 μm, inclusive). The vascular network and the triply periodic minimal surface can be separated by an interface distance. The interface distance can be in a range of 1 μm to 400 μm (e.g., 1 μm, 5 μm, 10 μm, 25 μm, 50 μm, 100 μm, 200 μm, 300 μm, 400 μm, inclusive).

The 3D scaffold can include one or more channels (e.g., airway channels, airways, etc.). The one or more channels can hold a gas (e.g., O₂). The one or more channels can be formed by the 3D scaffold. The vascular network can be embedded inside walls of the 3D scaffold. The one or more channels can be positioned between the walls of the 3D scaffold.

The method can include disposing the 3D scaffold into (e.g., in, on, onto, etc.) a gas exchange chip. For example, the method can include placing the 3D scaffold into a matrix of the gas exchange chip. The 3D scaffold can include a gas exchange scaffold seeded with biological materials. The gas exchange scaffold seeded with the biological materials can be disposed within the gas exchange chip.

Any references to implementations or elements or acts of the systems and methods herein referred to in the singular can include implementations including a plurality of these elements, and any references in plural to any implementation or element or act herein can include implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

Any implementation disclosed herein may be combined with any other implementation, and references to “an implementation,” “some implementations,” “an alternate implementation,” “various implementations,” “one implementation” or the like are not necessarily mutually exclusive and are intended to indicate that a particular feature, structure, or characteristic described in connection with the implementation may be included in at least one implementation. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Elements other than ‘A’ and ‘B’ can also be included.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

The systems and methods described herein may be embodied in other specific forms without departing from the characteristics thereof. The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

What is claimed is:

1. An apparatus comprising:
a triply periodic minimal surface;
a 3D scaffold formed from the triply periodic minimal surface; and
one or more channels formed by the 3D scaffold.

2. The apparatus of claim 1, further comprising:
a vascular network embedded within the 3D scaffold.

3. The apparatus of claim 2, wherein the vascular network has a thickness in a range of 5 μm to 500 μm.

4. The apparatus of claim 2, wherein the vascular network and the triply periodic minimal surface are separated by an interface distance in a range of 1 μm to 400 μm.

5. The apparatus of claim 1, further comprising:
a gas exchange chip;
wherein the 3D scaffold comprises a gas exchange scaffold seeded with biological materials; and
wherein the gas exchange scaffold seeded with the biological materials is disposed within the gas exchange chip.

6. The apparatus of claim 1, wherein the one or more channels comprise airways.

7. The apparatus of claim 1, wherein the triply periodic minimal surface comprises a gyroid.

8. The apparatus of claim 1, wherein the triply periodic minimal surface comprises a Batwing.

9. The apparatus of claim 1, wherein the triply periodic minimal surface comprises a Fischer S.

10. The apparatus of claim 1, wherein the 3D scaffold has a thickness in a range of 15 μm to 700 μm.

11. The apparatus of claim 1, wherein:
the 3D scaffold comprises a 3D printed scaffold; and
the 3D printed scaffold models at least one of a pancreas, a kidney, a lung, a liver, and bone.

12. The apparatus of claim 1, wherein the 3D scaffold comprises a cell bioreactor that is permeable to at least one of gas, fluid, small molecules, cytokines, or growth factors.

13. The apparatus of claim 1, wherein the triply periodic minimal surface is at least one of an internal mechanical support structure or an external mechanical support structure for a 3D printed scaffold.

14. A method of forming a gas exchange unit, comprising:
printing a 3D scaffold formed from a triply periodic minimal surface, the 3D scaffold comprising;
a vascular network configured to conduct a fluid; and
one or more channels configured to hold a gas;
wherein the vascular network is embedded inside walls of the 3D scaffold; and
wherein the one or more channels are positioned between the walls of the 3D scaffold.

15. The method of claim 14, further comprising:
disposing the 3D scaffold into a gas exchange chip.

16. The method of claim 14, wherein the triply periodic minimal surface comprises a gyroid.

17. The method of claim 14, wherein the triply periodic minimal surface comprises a Batwing.

18. The method of claim 14, wherein the triply periodic minimal surface comprises a Fischer S.

19. The method of claim 14, wherein the vascular network has a thickness in a range of 5 μm to 500 μm.

20. The method of claim 14, wherein the vascular network and the triply periodic minimal surface are separated by an interface distance in a range of 1 μm to 400 μm.
